(19) United States
(12) Patent Application Publication
(10) Pub. No.: US 2011/0054326 A1
(43) Pub. Date: Mar. 3, 2011

(54) DIRECT VISUALIZATION CATHETER

(75) Inventor: Michael A. Barnett, Franklin, TN (US)

(73) Assignee: VANDERBILT UNIVERSITY, Nashville, TN (US)

(21) Appl. No.: 12/819,926

(22) Filed: Jun. 21, 2010

Related U.S. Application Data
(60) Provisional application No. 61/218,468, filed on Jun. 19, 2009.

(51) Int. Cl. A61B 1/00; A61B 1/04; A61B 1/06; A61B 8/00

(52) U.S. Cl. 600/453; 600/109; 600/178; 600/104; 600/106; 600/113; 600/101

(57) ABSTRACT

An endoscopic device providing direct visualization of a surgical procedure, such as a surgical procedure in which there is significant body and/or artificially introduced fluids is provided. This endoscopic device comprises catheter with a lumen in which a transparent gel may be housed. During the surgical operation the transparent gel may be dispensed at the target site to displace the body fluids, such as blood. A visualization tool housed in the catheter may then be inserted into the gel to allow a surgeon to view the target site through the gel. Additionally, the catheter may comprise a surgical tool(s) that a surgeon may use to perform an operation at the target site. After completion of the operation, the gel and tools may be retracted back into the catheter and removed from the patient's body.

### CROSS-REFERENCE TO RELATED APPLICATIONS

This application claims the benefit from U.S. Provisional Application No. 61/218,468, entitled “Direct Visualization Catheter,” filed on Jun. 19, 2009. The contents of this application are hereby incorporated by reference herein.

### BACKGROUND

1. Field of the Invention

The present invention is generally directed to a catheter, and more particularly, to a catheter comprising a gel for direct visualization of a target site.

2. Related Art

Endoscopic or minimally invasive surgery, sometimes referred to as small incision surgery, is generally performed by inserting one or more diagnostic or surgical instruments into a patient's body through a relatively small incision(s). Generally, the endoscopic device includes a rigid or flexible tube (referred to as a catheter), a light delivery system to illuminate the tissue under inspection, and a camera system for transmitting an image of the tissue to the surgeon. Additionally, endoscopic devices may also some times include one or more medical tools, such as scissors, forceps, biopsy device, clamp, etc., disposed at the distal end of the tube. The tool(s) is typically coupled to a handle situated at the proximal end of the catheter which permits the surgeon to operate the tool.

The camera system typically relays images to a monitor external to the patient so that the surgeon may directly visualize the operation of any other diagnostic or surgical instruments also inserted into the patient. As used herein, direct visualization refers to viewing an image captured by a camera.

In certain operative circumstances, body fluids may be present at the tissue site that may prevent the surgeon from obtaining a clear view of the instruments and/or the tissue. One such example is intra-cardiac surgery in which an operation is performed on an interior portion of the heart where a significant amount of blood may be present. Or, in other situations, the surgeon's view may be obscured by other fluids which may be introduced into the operative field as part of the procedure, such as with irrigation, aqua-dissection, etc. It is thus difficult or impossible to use prior endoscopic devices in these situations where fluids may prevent or significantly inhibit direct visualization of the tissue.

### SUMMARY

In one aspect of the invention, there is provided a multi-lumen steerable endoscope, comprising: a catheter comprising: an imaging lumen; and a medium lumen; a visualization tool housed in said imaging lumen; and a gel housed in said medium lumen configured to be dispensed at least in part from said medium lumen and retracted back into said medium lumen.

In another aspect of the invention, there is provided a method for performing an operation using a multi-lumen steerable endoscope, comprising: inserting a catheter comprising an imaging lumen and a medium lumen into a patient's body; routing the catheter to a target site within the patient's body; dispensing a gel at the target site from the medium lumen; observing the target site using a visualization tool housed in said imaging lumen; retracting said gel back into said medium lumen after performance of the operation; and removing said catheter from said patient's body.

In another aspect of the invention, there is provided a catheter comprising: a medium lumen; and a gel housed in said medium lumen configured to be dispensed at least in part from said medium lumen and retracted back into said medium lumen.

### BRIEF DESCRIPTION OF THE DRAWINGS

Illustrative embodiments of the present invention are described herein with reference to the accompanying drawings, in which:

FIG. 1 is a perspective view of an exemplary endoscopic device in which embodiments of the present invention may be advantageously implemented;

FIG. 2 illustrates a simplified version of a human heart in which an endoscopic device is inserted, in accordance with an embodiment;

FIG. 3 illustrates a flow chart of a method for performing a surgical operation using an endoscopic device, in accordance with an embodiment;

FIG. 4A provides a simplified illustration of a gel dispensed at a target site, in accordance with an embodiment;

FIG. 4B provides a simplified illustration of a gel in which a tools have been inserted at a target site, in accordance with an embodiment;

FIG. 4C provides a simplified illustration of a target site after retraction of a gel, in accordance with an embodiment;

FIG. 4D is a simplified illustration of a gel having a support structure therein, in accordance with embodiments of the present invention;

FIG. 5 illustrates an endoscope that uses a pull-rod mechanism for dispensing and retracting a gel, in accordance with an embodiment; and

FIG. 6 illustrates a simplified version of a human heart in which a plurality of catheters are inserted, in accordance with an embodiment.

### DETAILED DESCRIPTION

Due to the presence of body and/or artificially introduced fluids, certain operative procedures within a patient's body have previously precluded the use of an endoscope to directly visualize the operative procedure. Exemplary such operative procedures include those involving a patient's cardiovascular system. For example, if using an endoscope in cardiovascular surgery, the blood distributed through the cardiovascular system can cover the camera lens or be present in such quantity between the endoscopic device and the target tissue that the fluids may prohibit or greatly degrade the ability of the surgeon to directly visualize the target tissue.

Embodiments of the present invention are generally directed to a catheter, such as a catheter for use in the presence of body and/or artificially introduced fluids. As will be discussed in further detail below, in an embodiment, a catheter comprises a transparent gel which may be dispensed during a medical procedure or surgery (collectively and generally referred to as “surgery” herein) at a target site. This gel may displace the body fluid from the target site allowing the light source of an endoscope to illuminate the target site, thereby enabling the endoscope to have a clear view of the target site. After surgery, the gel may be retracted back into the catheter, which is then removed from the patient. As will be discussed in further detail below, the catheter in which the gel is housed may be a catheter of an endoscope that also houses a visualization tool.

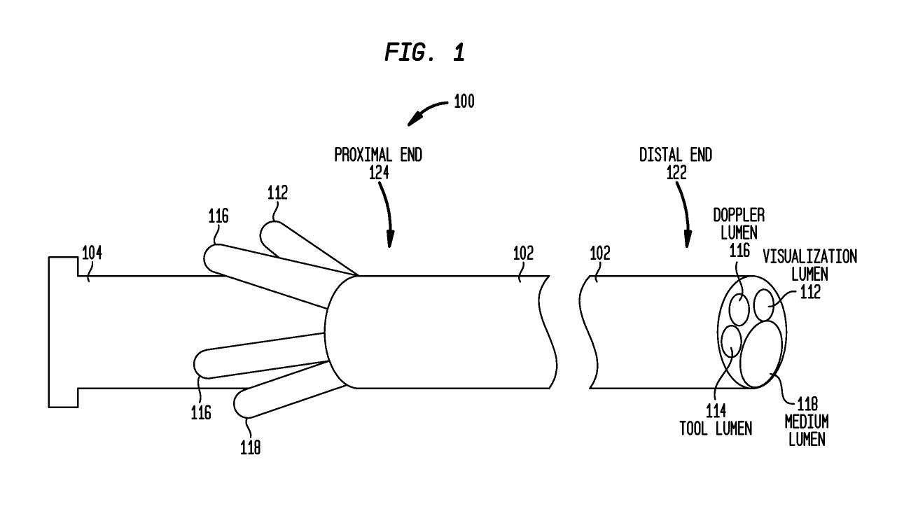

FIG. 1 illustrates an endoscopic device, in accordance with an embodiment. As illustrated, endoscopic device 100 comprises a catheter 102 and a handle 104. Catheter 102 may comprise a visualization lumen 112, a tool lumen 114, a Doppler lumen 116, and a medium lumen 118. Each of these lumens may extend through the length of the catheter 100. Further, catheter 102 may, for example, have an outer diameter of 6 mm/18 French, and a cross-sectional area of 28.26 mm². Additionally, catheter 102 may be a multi-lumen steerable catheter such as a catheter similar to the catheter described in U.S. Pat. No. 7,037,290 to Gardeski et al., filed on Dec. 16, 2002, which is hereby incorporated by reference in its entirety.

Visualization lumen 112 may be used to enable a camera and light source to extend through catheter 102. The camera and light source extending through lumen 112 may be any type of camera and light source such as commonly used in endoscopes. For example, in an embodiment the light source and camera may be an Olympus BF-2.2 T fiber optic light source and scope having an outer diameter of 2.2 mm. Further, the use of cameras and light sources in endoscopes is well known to those of skill in the art, and as such is not described further herein.

Tool lumen 114 may be used to enable a tool to extend through catheter 102. This tool may be any tool useable in endoscopic surgery, such as, for example, a suture tool, a cautery tool, a scalpel, forceps, scissors, a biopsy device, a clamp, etc. Further, in an embodiment, tool lumen 114 may have a diameter such as, for example, 2.8 mm. Doppler lumen 116 may be used to enable a Doppler device to extend through catheter 102. Doppler devices may be used for detection of blood flow in facilitating catheter placement. The use of tools and Doppler devices in an endoscopic device are well known to those of skill in the art, and as such are not described further herein. In an embodiment, each of lumens 112, 114, and 116 may have the same configuration and any type of appropriately configured tool (e.g., a visualization tool, Doppler tool, or surgical tool) may be inserted through lumens 112, 114, and 116. Accordingly, each of these lumens may also be referred to generically as a tool lumen.

Medium lumen 118 may be used to house a transparent gel. This transparent gel may be extended out of catheter 102 during surgery and then retracted back into catheter 102 after completion of the surgery. This transparent gel may enable light from the light source to pass through the gel to illuminate the target site, and allow camera to visualize the illuminated target site. This transparent gel may be clear in color, or in other embodiments may have a different color, such as for example, a yellowish tint color. The gel further may have low surface friction to enable gel to easily be pushed out of and retracted back into medium lumen 118. Further, this gel may also have a sufficiently low modulus so that when extended out of lumen 118, the gel may conform with the surface of the target site. Additionally, the gel may have sufficient tear strength to reduce the possibility of a piece of gel tearing off during the operation. Further, gel also may have self healing properties so that tools, such as surgical and visualization tools, may be inserted into gel and removed without leaving a significant witness line or other marks which may reduce the visibility through the gel.

In an embodiment, medium lumen 118 may have a larger diameter at the distal end 122 of catheter 102 than at the proximal end 124, and one or more or all lumens may be collapsible at their distal ends 122 to provide additional space for allowing the gel to have a wide neck when dispensed. The proximal ends of lumens in contrast may have a fixed diameter.

Further, the gel may only be housed in the distal end 122 of catheter 102 (i.e., the end opposite handle 104) and be bonded to a tool that a surgeon may use to dispense and retract the gel from lumen 118. For example, in an embodiment, the gel may be bonded to a push rod that a surgeon may push to dispense the gel, and then pull back to retract the gel back into lumen 118. Or, in another embodiment, the gel may be bonded to a rod connected to a ratchet mechanism located at the proximal end 124 of endoscopic device 100, such as on handle 104. A surgeon may then use the ratchet mechanism to dispense the gel, and then retract the gel. In still other embodiments, the gel may be dispensed or retract through the use of a vacuum or through the use of hydrostatic pressure. Such embodiments are described further below

It should be noted that the number and type of lumens included in catheter 102 is exemplary, and, in other embodiment, catheter 102 may include more or less lumens. For example, in embodiments, catheter 102 may include a plurality of tool lumens each for a different tool. Or, for example, other types of devices may be used to extend through the lumens of catheter 102. Or, in yet another embodiment, catheter 102 may include only a single lumen, such as medium lumen 118 housing the transparent gel as well as the mechanism for dispensing and retracting the gel. Such a catheter may then be used along with a separate endoscope to permit a surgeon to directly visualize a target site through gel dispensed from the catheter during an operation.

Additionally, in certain embodiments the catheter may be configured such that gel may be dispensed from a plurality or all of the lumens within the catheter. In other embodiments, it is not necessary to have a dedicated medium lumen. In these embodiments, a lumen may serve multiple functions to dispense/retract the gel, as well as to provide access for tools, light sources, cameras, etc. In a specific such embodiment, the catheter comprises a single lumen which serves multiple functions, including dispensing and retraction of the gel.

Endoscopic device 100 further comprises a handle 104 that may be used to steer the catheter 102 to the target site. Additionally, handle 104 may comprise the proximal ends 124 of lumens 112, 114, 116 to allow the passage of devices, such as a light source/camera, a Doppler device, and tools through the lumens

Handle 104 may comprise a mechanism for allowing a surgeon to dispense gel from the medium lumen 118 at the target site during a procedure, and then retract the gel back into the medium lumen 118. Further descriptions of exemplary mechanisms for dispensing and retracting the gel are provided in more detail below.

The below described embodiment will be discussed primarily in the context of endoscopic device 100 used in endovascular intracardiac surgery to perform an operation on the right ventricle of a patient's heart. However, it should be understood that exemplary catheters in accordance with the present invention may used in other operative procedures, such as, for example, lead placement, vegetation removal, Atrial septal defect/Ventricular septal defect (ASD/VSD) closure, inspection and therapy of prosthetic valves, pericardial fluid sampling, endomyocardial bypass, etc.

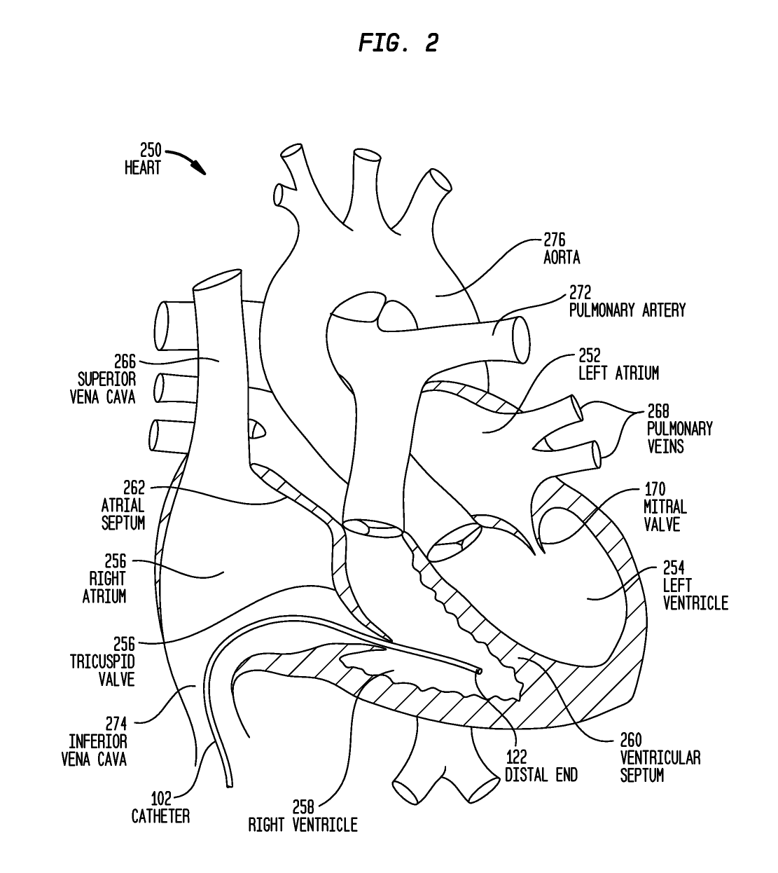

FIG. 2 illustrates a simplified version of a human heart in which an endoscopic device is inserted for performing surgery, in accordance with an embodiment. As illustrated, heart 250 comprises four separate chambers known as the left atrium 252, left ventricle 254, right atrium 256 and right ventricle 258. Ventricles 254 and 258 are separated by the ventricular septum 260, while atria 256 and 252 are separated by atrial septum 262.

Oxygen poor blood enters heart 250 through two large veins, superior vena cava 266 and inferior vena cava 274 into right atrium 256. The blood flows from right atrium 256 into right ventricle 258 through an open tricuspid valve 264. Meanwhile, pulmonary veins 268 empty oxygen-rich blood from the lungs (not shown) into left atrium 252. The oxygen rich blood flows from left atrium 252 into left ventricle 254 through open mitral valve 270.

When the ventricles are full, tricuspid valve 264 and mitral valve 270 shut to prevent blood from flowing backward into atria 252 and 256 during ventricular contraction. Ventricular contraction causes oxygen poor blood to flow from right ventricle 258 through pulmonary artery 272 to the lungs, where the blood is oxygenated and then returned to left atrium. Ventricular contraction also causes oxygen rich blood to leave left ventricle 254 through aorta 276 to the remainder of the body. This pattern is repeated over and over, causing blood to flow continuously to heart 250, the lungs and the remainder of the body.

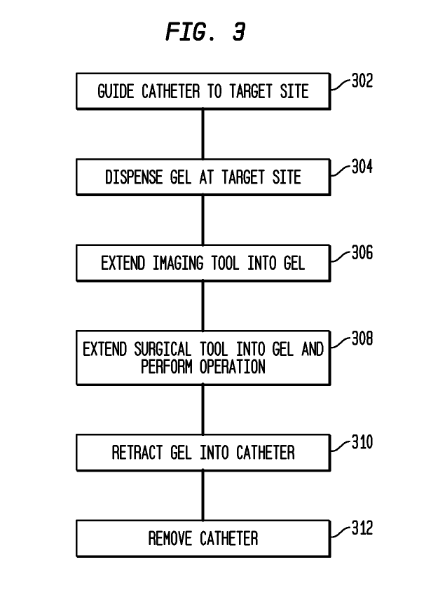

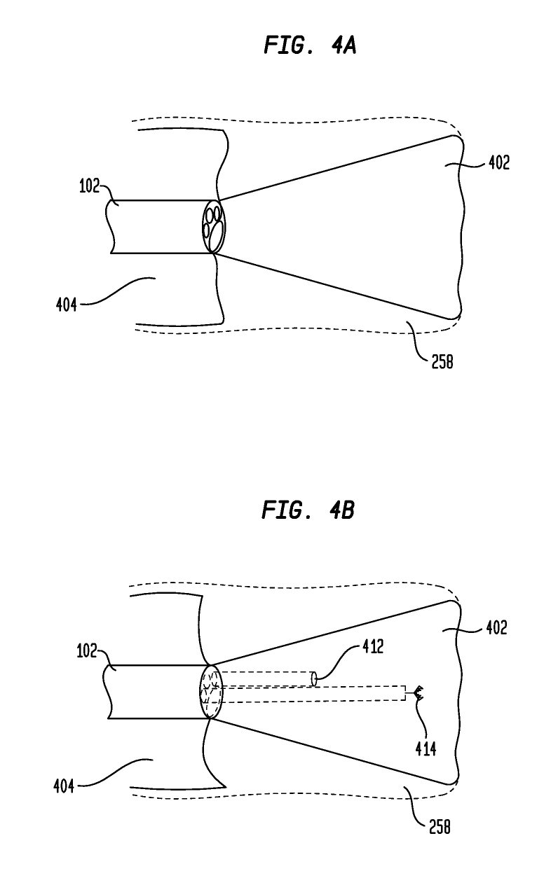

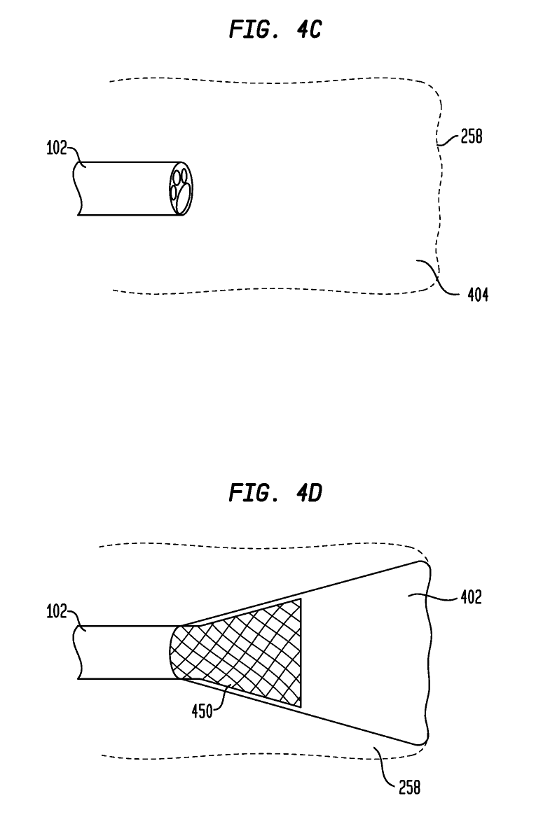

FIG. 3 illustrates a flow chart of method for performing a surgical operation using an endoscopic device, in accordance with an embodiment. FIG. 3 will be discussed with reference to FIGS. 2 and 4A-4C, which illustrate a simplified close up view of the target site in various stages of the method of FIG. 3.

During surgery, endoscopic device 100 may be surgical inserted through an incision in the patient's skin and extended through inferior vena cava 274 into heart 250. As shown in FIG. 2, catheter 102 extends through right atrium 256 and tricuspid valve 264 such that a distal end 122 of catheter 102 is positioned in right ventricle 258. In this example, right ventricle 258 is the target site (hereinafter referred to as target site 258). Catheter 102 may be routed to the target site using traditional fluoroscopy and/or auditory input from a Doppler device housed in Doppler lumen 116. Routing an endoscopic device to a target site is well know to those of skill in the art, and as such is not described further herein.

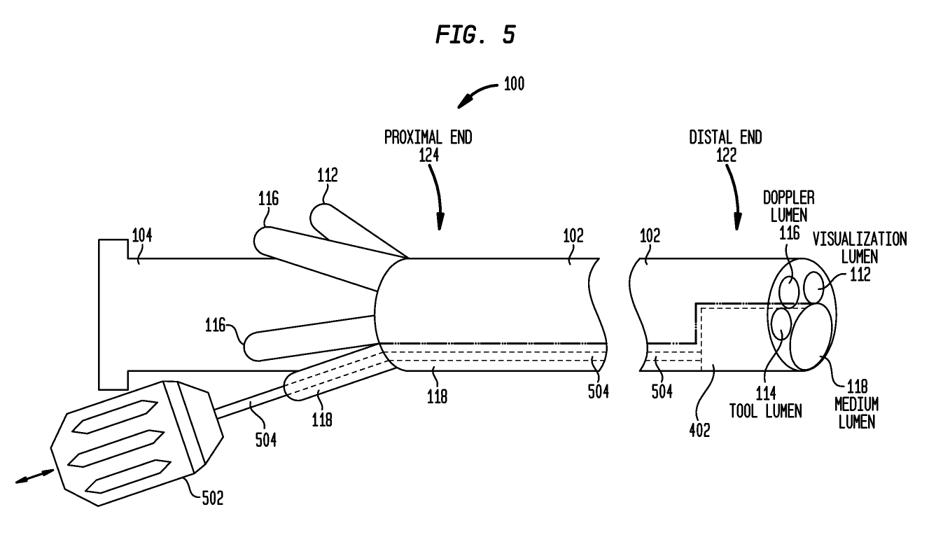

Once endoscopic device 100 is positioned at the target site, the surgeon may cause the gel housed in medium lumen 118 to be dispensed at the target site 258 at block 304. As noted above, endoscopic device 100 may use various mechanisms for dispensing the gel 402. In one example, the gel 402 may be bonded within medium lumen 118 to a push rod that a surgeon may use to dispense and retract the gel 402. Or, for example, a ratchet mechanism may be used for dispensing and retracting the gel 402. In other embodiments, gel 402 may be dispensed and retracted via vacuum or hydrostatic pressure. A further description of exemplary mechanisms for dispensing gel using a push rod is provided below with reference to FIG. 5.

FIG. 4A illustrates a simplified illustration of a gel 402 dispensed at a target site 258, such as ventricle 258 of FIG. 2. Gel 402 may be optically clear and allow light to pass through it so that the target site may be illuminated by the light source and visible to the camera thus allowing the surgeon to have direct visualization of the target site 258. Gel may further exhibits gel like properties at body temperature, such that it is deformable and may be conformed to the shape of the target site 258. For example, during surgical operations, a surgeon may dispense gel 402 at the target site 258 such that gel 402 makes direct contact with target site 258 and displaces any body fluids 404, such as blood from the target site 258.

Gel 402 may further have properties such that it exhibits low surface friction with medium lumen 118 such that the gel 402 may easily be dispensed from and retracted into lumen 118. Gel 402 may also have sufficient tear strength and toughness so that gel 402 maintains its integrity during surgical operations so that a portion of gel 402 does not become detached during the surgery. Gel 402 also preferably is made of a non-toxic material. Gel 402 may be formed from a number of different materials exhibiting the desired properties. In certain embodiments, gel 402 is a silicone, while in other embodiments a urethane gel may be used.

Additionally, in an embodiment, gel 402 may also be self-healing so that tools, such as surgical and visualization tools may be inserted into and removed from gel 402 without leaving a witness line or leaving a minimal witness line. This may be beneficial in enabling a surgeon to insert and move tools in gel 404 while not significantly impairing the visibility of the target site 258 by the visualization tool.

After dispensing gel 402, a surgeon may insert a visualization tool into gel 402 at block 306. The surgeon may use the visualization tool to observe the target site 258 during, for example, performance of the procedure. The surgeon may also dispense a surgical tool at block 308 to perform a surgical procedure. FIG. 4B illustrates a simplified illustration of a gel 402 dispensed at a target site 258 in which tools have been inserted, in accordance with an embodiment. As illustrated, a visualization tool 412 housed in visualization lumen 112 and a surgical tool 414 housed in tool lumen 114 may be inserted into gel 402. As noted above, visualization tool 412 may comprise a light source and a camera, such as, for example, visualization tools commonly used in endoscopes. Further, surgical tool 414 may be any type of tool useful in performing an operation on a patient.

After performing the surgical operation, the surgeon may retract the gel 402 back into medium lumen 118 at block 310. FIG. 4C illustrates a simplified illustration of a target site after retraction of the gel, in accordance with an embodiment. As illustrated, after retraction of the gel, the body fluid may return to the space previously occupied by gel 402.

After retraction of gel 402, the surgeon may then remove catheter 102 from the patient's body at block 312. Catheter 102 may be removed from the patient's body using any suitable mechanism. Removal of a catheter after performance of a surgical operation is well known to those of skill in the arts, and as such is not described further herein.

Furthermore, in certain embodiments of the present invention, gel 402 may have a support structure disposed therein. FIG. 4D illustrates the use of one such support structure 450. In this embodiment, support structure 450 comprises a mesh that is dispensed from catheter 102. Mesh 450 may dispensed in a conical form and provides a structure for gel 402 to adhere to and to facilitate effective dispensing and retraction of the gel. Mesh 450 may be formed of metal fibers, such as nitinol. It would be appreciated that other support structures may be implemented in accordance with embodiments of the present invention and the embodiment of FIG. 4D is merely exemplary.

FIG. 5 illustrates an embodiment of endoscope 100 that uses a pull rod mechanism for dispensing and retracting the gel, in accordance with an embodiment. As illustrated, gel 402 may be include in a wider portion of medium lumen 118 located at the distal end 122 of catheter 102. The gel 402 may be bonded to a rod 504 using any suitable means. Rod 504 may pass through lumen 118 to handle 502. A surgeon may then push the handle 502 toward the distal end 122 of the catheter 102 to dispense the gel 402, and pull the handle 504 in the opposite direction to retract the gel 402. Further, handle 502 or rod 504 may include markings to help indicate whether the gel is fully dispensed or retracted. It should be noted that this is a simplified diagram provided to illustrate how a pull rod mechanism may be used to dispense and retract gel, and other components may be included in endoscopic device.

In another embodiments, the rod 502 and handle 504 may traverse down the center of handle 104, and or handle 502 may be used in place of handle 104. Further, although in this simplified diagrams, lumens 112, 114, and 116 are not illustrated as connected to a handle or other device on their proximal end, it should be noted that these proximal ends may be connected to a handle or controller as appropriate for the type of tool included in the respective lumen.

In another embodiment, rather than using a pull-rod for dispensing and retracting gel 402, a ratchet mechanism may be used. This ratchet mechanism may be connected to a rod, such as rod 504 bonded to gel 402 for dispensing and retracting the gel 402. Further, this ratchet mechanism may be included at any appropriate location at the proximal end of the endoscope, such as, for example, on handle 104.

The methods or dispensing and retraction of gel 402 described with reference to FIG. 5 are merely illustrative and it would be appreciated that other methods are within the scope of the present invention. For example, in one embodiment, gel 402 may be dispensed and/or retracted using a vacuum. In such embodiments, a vacuum system may connected to the catheter to use air pressure to dispense or retract gel 402. In other embodiments, hydrostatic pressure may be used to dispense/retract gel 402. That its, a liquid may be used to exert push/pull forces on gel 402.

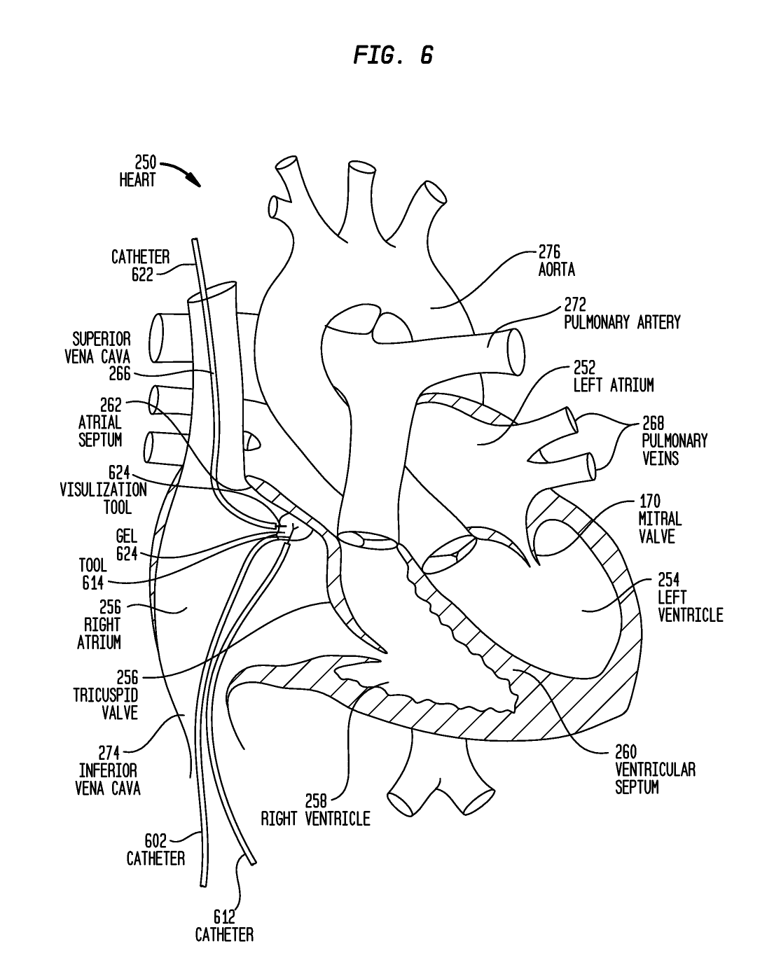

FIG. 6 illustrates a simplified version of a human heart in which a catheter including a gel is inserted for performing surgery, in accordance with an embodiment. In this exemplary embodiment, three catheters 612, 622, and 624 are used to perform an operation, such as a transseptal puncture in the atrial septum 262 of the patient's heart 250. Catheter 612 may be a catheter configured to dispense and detract a transparent gel 604 to the target site, which in this case is atrial septum 262. Catheter 612 may have a single lumen housing gel 604 and a mechanism for dispensing and retracting the gel, such as those discussed above.

Catheter 622 may be, for example, a catheter for an endoscope comprising a visualization tool 624. Or, in other embodiments, catheter 612 may comprise multiple lumens, such as, for example, a lumen for housing visualization tool 624 in addition to a lumen for housing gel 604. Catheter 612 may comprise, for example, a single lumen housing a surgical tool 614 for use in performing the operation. For example, tool 614 may be a myocardial rivet. Or, in other embodiments, catheter 622 may comprise multiple lumens housing other tools, such as, for example, forceps, etc.

In performing the operation, a surgeon may route each of catheters 602, 612, and 622 to atrial septum 262 from the same or different access locations. Then, the surgeon may dispense gel 604 from catheter 602 using a mechanism, such as the above-discussed pull rod mechanism of FIG. 5. The surgeon may dispense gel 604 so that a slight pressure is applied against the atrium septum 262 by gel 604 that causes gel 604 to mold itself to the shape of atrium septum 262. This may cause gel 604 to spread out in a direction perpendicular to the longitudinal direction of catheter 602.

The surgeon may then insert visualization tool 614 from catheter 612 into gel 604 to provide the surgeon with direct visualization of the target site. Then, the surgeon may perform the operation using tool 624. As noted above, gel 604 may have self healing properties that allow the surgeon to move tools 614 and 624 around gel 604 without significantly damaging gel 604 (e.g., without leaving significant witness lines) and impairing the visualization of the target site.

After completion of the surgery, the surgeon may retract tools 614 and 624 from gel 604. Then, the surgeon may retract gel 604 back into catheter 602. After which, the surgeon may remove catheters 602, 612, and 614 from the patient's body.

In embodiments, the catheters, such as the catheters discussed above, may have rapid exchange capabilities. For example, in an embodiment, a plurality of catheters may be manufactured, each comprising a gel and a mechanism for dispensing the gel, such as discussed above. Each of these catheters may have a rapid exchange capability. Further, tools, such as those discussed above (e.g., a visualization tool, myocardial rivet, a Doppler tool, etc.) may be manufactured such that they are configured to work with each of the plurality of catheters. Further, the catheters may be manufactured such that these tools may be exchanged between the catheters using their rapid exchange capability. This may enable the tools to be reusable in different catheters. For example, in an embodiment, a first catheter, such as catheter 100 with rapid exchange capability may be used during a first procedure where a visualization tool, a surgical tool, and a Doppler device are inserted through lumens 112, 114, and 116. Then, after the operation one or more of these tools may be detached from the catheter, sterilized, and attached to a new catheter for subsequent use in another operation.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

What is claimed is:

1. A multi-lumen steerable endoscope, comprising:
a catheter comprising:
an imaging lumen; and
a medium lumen;
a visualization tool housed in said imaging lumen;
a gel housed in said medium lumen configured to be dispensed at least in part from said medium lumen and retracted back into said medium lumen.

2. The multi-lumen steerable endoscope of claim 1, wherein said visualization tool comprises a camera.

3. The multi-lumen steerable endoscope of claim 2, wherein said visualization tool comprises a light source.

4. The multi-lumen steerable endoscope of claim 1, wherein said catheter further comprises a tool lumen, the endoscope further comprising:
a surgical tool housed in said tool lumen.

5. The multi-lumen steerable endoscope of claim 1, wherein said catheter further comprises a Doppler lumen, the endoscope further comprising:
a Doppler device.

6. The multi-lumen steerable endoscope of claim 1, further comprising:
a mechanism at least partially housed in said medium lumen for dispensing and retracting said gel.

7. The multi-lumen steerable endoscope of claim 6, wherein the mechanism comprises:
a rod connected to said gel; and
a handle connected to a pull-rod that may be manipulated to dispense and retract said gel.

8. The multi-lumen steerable endoscope of claim 6, wherein the mechanism comprises:
a rod connected to said gel; and
a ratchet mechanism connected to said rod that may be manipulated to dispense and retract said gel.

9. The multi-lumen steerable endoscope of claim 1, wherein the gel comprises:
a silicone gel.

10. The multi-lumen steerable endoscope of claim 1, wherein the gel is a transparent gel.

11. The multi-lumen steerable endoscope of claim 1, wherein the gel is dispensed and retracted using vacuum pressure.

12. The multi-lumen steerable endoscope of claim 1, wherein the gel is dispensed and retracted using hydrostatic pressure.

13. A method for performing an operation, comprising:
inserting a catheter comprising a medium lumen into a patient's body;
routing the catheter to a target site within the patient's body;
dispensing a gel at the target site from the medium lumen;
observing the target site using a visualization tool;
retracting said gel back into said medium lumen after performance of the operation; and
removing said catheter from said patient's body.

14. The method of claim 13, wherein the catheter further comprises an imaging lumen housing the visualization tool, and wherein observing said target site comprises:
observing said target site using a visualization tool comprising a camera.

15. The method of claim 14, wherein said visualization tool comprises a light source.

16. The method of claim 13, further comprising:
performing an operation at the target site using a surgical tool housed in a tool lumen included in said catheter.

17. The method of claim 13, wherein routing said catheter to said target site comprises:
routing said catheter using a Doppler device housed in a Doppler lumen included in said catheter.

18. The method of claim 13, wherein dispensing and retracting said gel comprises:
dispensing and retracting said gel using a mechanism at least partially housed in said medium lumen.

19. The method of claim 18 wherein the mechanism comprises a rod connected to said gel and a handle connected to the rod, and wherein dispensing said gel comprises:
pushing the handle towards a distal end of the catheter to dispense said gel; and
wherein retracting said gel comprises:
pulling the handle away from said distal end to retract said gel.

20. The method of claim 18, wherein the mechanism comprises a rod connected to said gel and a ratchet mechanism connected to said rod, and wherein dispensing said gel comprises:
manipulating the ratchet mechanism to dispense said gel; and
wherein retracting said gel comprises:
manipulating the ratchet mechanism to retract said gel.

21. The method of claim 18, wherein the gel comprises:
a silicone gel.

22. The method of claim 13, wherein dispensing the gel comprises:
dispensing the gel using vacuum pressure.

23. The method of claim 13, wherein dispensing the gel comprises:
dispensing the gel using hydrostatic pressure.

24. The method of claim 13, further comprising:
inserting a second catheter into the patient's body; and
observing the target site using a visualization tool of the second catheter.

25. A catheter comprising:
a medium lumen; and
a gel housed in said medium lumen configured to be dispensed at least in part from said medium lumen and retracted back into said medium lumen.

26. The catheter of claim 25, further comprising:
an imaging lumen; and
a visualization tool comprising a camera housed in said imaging lumen.

27. The catheter claim 21, wherein said visualization tool comprises a light source.

28. The catheter of claim 25, further comprising:
a tool lumen; and
a surgical tool housed in said tool lumen.

29. The catheter of claim 25, further comprising:
a Doppler lumen; and
a Doppler device housed in said Doppler lumen.

30. The catheter of claim 25, further comprising:
a mechanism at least partially housed in said medium lumen for dispensing and retracting said gel.

31. The catheter of claim 25, wherein the mechanism comprises:
a rod connected to said gel; and
a handle connected to a pull-rod that may be manipulated to dispense and retract said gel.

32. The catheter of claim 25, wherein the mechanism comprises:
a rod connected to said gel; and
a ratchet mechanism connected to said rod that may be manipulated to dispense and retract said gel.

33. The catheter of claim 25, wherein the gel comprises:
a silicone gel.

34. The catheter of claim 20, wherein the gel is a transparent gel.
